(19) United States
(12) Patent Application Publication
(10) Pub. No.: US 2011/0023879 A1
(43) Pub. Date: Feb. 3, 2011

(54) Ventilator Based On A Fluid Equivalent Of The "Digital To Analog Voltage" Concept

(75) Inventors: Joseph Douglas Vandine, Manteca, CA (US); Ravikumar Venkata Kudaravalli, Manassas, VA (US)

Correspondence Address: NELLCOR PURITAN BENNETT LLC, 6135 GUNBARREL AVENUE, BOULDER, CO, 80301, US

(73) Assignee: Nellcor Puritan Bennett LLC, Boulder, CO (US)

(21) Appl. No.: 12/935,824

(22) PCT Filed: Mar. 30, 2009
(86) PCT No.: PCT/US09/38816

§ 371 Date: Sep. 30, 2010

Related U.S. Application Data
(60) Provisional application No. 61/041,099, filed on Mar. 31, 2008.

(51) Int. Cl. A61M 16/00

(52) U.S. Cl. 128/204.21

(57) ABSTRACT

The present invention is directed to a ventilator that, in one embodiment, uses one or more valve banks having precalibrated orifices to perform real time control of flow metering devices and, in a second embodiment, uses a choked flow orifice and upstream gas pressure regulator to generate a desired flow trajectory.

### FIELD

The invention relates generally to respiratory devices and particularly to mechanical ventilators.

### BACKGROUND

A medical ventilator is an automatic machine designed to mechanically move breathable air into and out of the lungs and thereby provide respiration for a patient. A typical ventilator includes air and/or oxygen sources, a set of valves and tubes, and a disposable or reusable patient circuit. During an inspiration phase, pressurized air or an oxygen/air mixture is provided to the patient. In the expiration phase, the overpressure is released, causing the patient to exhale.

There are several techniques to provide the pressurized air or oxygen/air mixture of a selected oxygen composition (e.g., FiO2) to the patient. In one ventilator configuration, each gas source is pressurized and has a proportional solenoid (PSOL) valve to control selectively and independently flow from the gas source, thereby providing a selected Fi02. In another ventilator configuration, a turbine or blower is employed to pressurize and meter the air flow. A controlled flow rate of oxygen is introduced into the blower intake or into the pressurized air downstream of the blower, thereby providing the selected Fi02. In another ventilator configuration, a piston pneumatically pressurizes the air. Controlled amounts of oxygen are introduced into the input to or output from the piston to realize the selected Fi02.

Existing ventilators can have a limited capability to define flow trajectory (or the flow as a function of time), realize the trajectory through complex means, or lack redundancy in the event of malfunction. Existing ventilators allow the user to specify a target for Fi02 (or the fraction of inspired oxygen in a gas mixture) but some maintain the specified Fi02 target constant for the entire breath cycle. Ventilators based on PSOL valve technology, turbine/blower, or piston-cylinder technology, can vary the specified Fi02 during the breath cycle but generally require sophisticated and dedicated closed loop controls. If a PSOL valve malfunctions, the composition of the inspired air can, depending on whether the malfunctioning PSOL valve operates on the air or molecular oxygen source, have an unacceptably low or high air or oxygen content. If a turbine, blower or piston fails, no pressurized gas is provided to the patient.

Another operational issue for ventilators is to accommodate patients of differing lung capacities. A premature infant, for instance, has a much smaller lung capacity than an adult. To address this issue, separate ventilators have been provided for infants and adults.

An example of an infant or pediatric ventilator is the Infant Star™ manufactured by Nellcor Puritan Bennett. This ventilator is time-cycled and pressure-limited and provides a continuous flow. The ventilator has air and oxygen sources, each metered by a separate valve, a mixing chamber, and a bank of solenoid valves downstream of the mixing chamber. The number of solenoid valves in the bank is selected based on a desired flow rate step, and the orifice sizes of the valves are related to the flow rate step. As the pressure in the mixing chamber drops, the metering valves open proportionately to recharge the chamber. The solenoid valve bank meters the flow from the mixing chamber to the patient circuit at a selected, but constant rate, by opening the appropriate combination of valves to deliver the desired flow

### SUMMARY

The present invention is directed generally to ventilators capable of defining desired gas composition and/or flow trajectories and servicing patients having widely differing lung capacities.

In a first embodiment, a ventilation method is provided that includes the steps:

(a) providing a ventilation system for receiving input gas(es) from one or more gas source(s), the ventilation system including one or more valve bank(s) to meter a flow of the input gas(es) and deliver an output gas to a patient, the valve bank(s) including a number of valves with each valve including an orifice;

(b) receiving a set of ventilation parameters;

(c) based on the set of ventilation parameters, determining, for each of a number of successive time intervals in an inspiration cycle, a number of operating states for selected valves in the valve bank(s) to provide the output gas, the output gas having one or more of a selected gas composition and flow trajectory; and

(d) when an inspiration cycle is initiated, implementing, for each successive time interval, the respective operating states for the selected valves in the valve bank(s).

This embodiment can provide a number of advantages over conventional ventilators. For example compared to existing trajectory shaping ventilators, the ventilator can simultaneously deliver any arbitrary flow trajectory and/or Fi02 trajectory with relatively simple pneumatics, controls, and electronics while enhancing performance and reliability and reducing costs. The ventilator, for example, can provide an FiO2 trajectory that is Fi02 100% at the beginning of inspiration and tapers off to Fi02 21% towards the end of inspiration. Thus, the ventilator can improve patient oxygen intake while reducing overall oxygen consumption. The ventilator can be robust. If a valve in the valve bank fails, the ventilator can still provide gas compositions and flow rates acceptable for most patients.

In a second embodiment, a ventilation method is provided that includes the steps:

(a) providing a ventilator for receiving input gas(es) from one or more gas sources and delivering an output gas for patient inhalation, the ventilator including one or more gas regulators to control a pressure of the input gas(es) and a valve positioned downstream of the gas regulator(s), the valve including an orifice; and

(b) while maintaining the valve at choked flow, varying the input gas pressure to provide differing output gas flow rates, such as for differing patients having differing lung capacities.

This embodiment can enable a common ventilator to service both adult and infant patients. Choked flow conditions permit the mass flow rate through the valve to be changed simply by changing the regulator's pressure set point.

These and other advantages will be apparent from the disclosure of the invention(s) contained herein.

### BRIEF DESCRIPTION OF THE DRAWINGS

FIG. 1 is a block diagram showing a ventilator according to an embodiment of the present invention;

FIG. 2 is a partial sequence of combinations of valve states according to an embodiment of the present invention;

FIG. 3 is a flowchart according to an embodiment of the present invention;

FIG. 4 is a plot of flow rate (SLPM) (vertical axis) versus time (seconds) (horizontal axis); and

FIG. 5 is a plot of flow rate (SLPM) (vertical axis) versus time (seconds) (horizontal axis).

### DETAILED DESCRIPTION

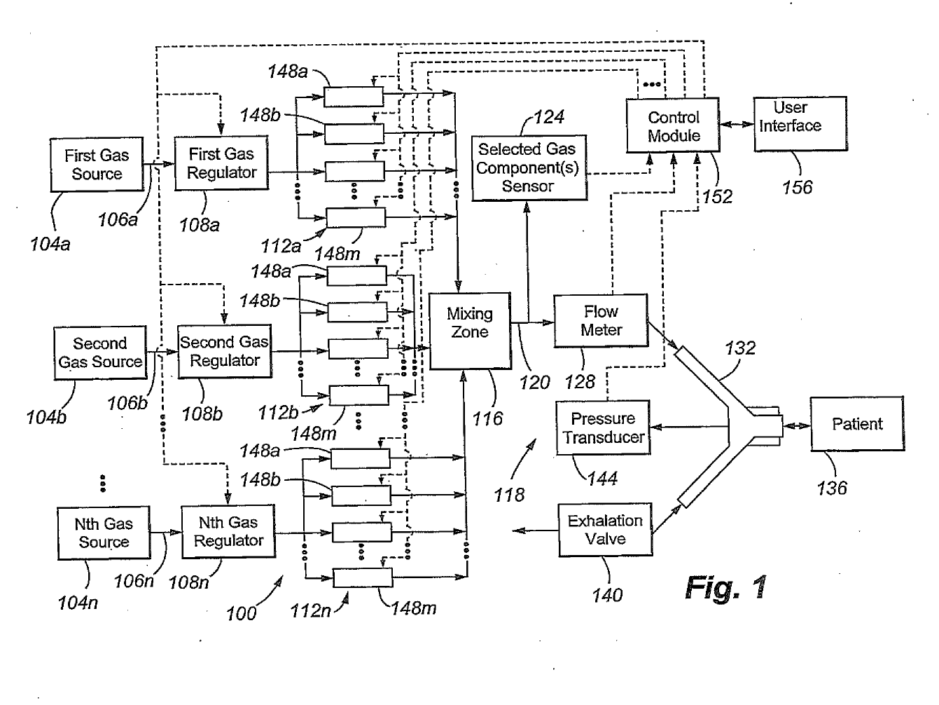

FIG. 1 depicts a ventilator system 100 according to a first embodiment. The ventilator system 100 can be any mechanical ventilator, including, without limitation, a bi-level breathing device. Input gases from the first, second, . . . nth gas sources 104a-n flow into the ventilator system 100 via conduits 106a-n. In the ventilator system 100, the input gases flow through corresponding first, second, . . . nth gas regulators 108a-n and into corresponding first, second, . . . nth valve banks 112a-n. The various gas flows outputted by the first, second, . . . nth valve banks 112a-n discharge into a mixing zone 116, where they form a substantially homogenous gas mixture 120. The output gas mixture 120 is then provided to a patient circuit 118 for delivery to a patient 136.

In the patient circuit 118, the output gas mixture 120 is sampled by a selected gas component inspiration sensor 124 and passed through an inspiration flow meter 128 and into an input branch of the patient wye 132. The wye 132 and associated conduits and other patient interface devices (not shown) provide the gas mixture to the patient 136. The exhaled gas is directed by the output branch of the wye 132 to an exhalation valve 140, which discharges the exhaled gas from the system 100. A pressure transducer 144 is in fluid communication with the input branch of the wye 132 and determines the pressure drop over the first, second, . . . , nth valve banks 112a-n. As will be appreciated, the patient circuit 118 can have other configurations and include fewer, different, and/or other components depending on the application.

The gas sources 104a-n are pressurized and can have any desired composition. In one configuration, the system 100 has only first and second gas sources 104a-b, one of which is predominantly molecular oxygen and the other of which is predominantly air. In yet another configuration, the system 100 has only one gas source 104a, which is predominantly either air or molecular oxygen. The gas source is typically a pressurized tank or other suitable source of pressurized gas, such as a gas delivery system found in a health care setting (e.g., compressed or wall air). In an alternative embodiment, the system 100 includes one or more compressors for compressing a gas, such as air, prior to delivery to a patient.

The first, second, . . . nth gas pressure regulators 108a-n can be any suitable arrangement for controlling the pressure of the respective gas upstream of the first, second, . . . nth valve banks 112a-n. Examples of suitable arrangements include a poppet, solenoid, butterfly, rotary, or sleeve valve. The outputs of the pressure regulators 108a-n are maintained to within a specified tolerance of a design pressure.

The mixing zone 116 is configured to provide adequate mixing of the various gas components received from the gas sources 104a-n. The mixing zone 116 can be any enclosed area, such as a vessel, a conduit, and the like. While FIG. 1 depicts a single mixing zone 116, in alternative embodiments more than one zone 116 may be used.

The first, second, . . . nth valve banks 112a-n each comprise a plurality of mechanically, electrically, pneumatically, hydraulically, magnetically, electromechanically or otherwise actuated valves 148a-m. At least some, or alternatively each valve has an orifice calibrated to deliver a specific flow rate for given design input and output pressures and binary operating states, namely an ON state and an OFF state. Preferably, the valves are two-way solenoid valves.

In one configuration, the number “m” of valves 148 in each valve bank 112 is selected based on a desired smallest flow rate step hereinafter referred to as the least significant bit (LSB) in analogy to digital electronics. The smallest valve's orifice is commonly calibrated for a flow rate of maximum flow rate/2^{m}. The maximum flow rate can be for the particular valve bank 112a-n, for the entire ventilator system 100, or both. For a maximum flow rate of 100 standard liters per minute (SLPM) and 8 solenoid valves in a valve bank, the LSB is 100 SLPM/2⁸=0.391 SLPM. As will be appreciated, other techniques for determining the orifice size(s) may be employed. The number m of valves 148a-m in a given valve bank 112a-n depends on the desired LSB for the valve bank 112a-n.

For given design input and output pressures, the valve 148 orifices in each valve bank 112a-n may be calibrated to deliver the same or different flow rates. When configured to provide different flow rates, the flow rates are preferably multiples of the LSB. For example, assuming that the LSB is X, a first valve 148a in the first valve bank 112a will deliver X, a second valve 148b in the first valve bank 112b 2X, a third valve 148c 4X, a fourth valve 148d 8X, . . . and nth valve 148m 2^{m}X. Other multipliers and orifice sizing schemes may be employed depending on the application.

The first, second, . . . nth valve banks 112a-n can have the same or differing characteristics. For example, the valve banks 112a-n can have the same or differing numbers of valves 148a-m. In another example, each of the valve banks 112a-n can be designed either to provide a common maximum flow rate Y and contain identically calibrated orifices or to provide different maximum flow rates and contain differently calibrated orifices. In the latter configuration, each of the differing valve banks 112a-n will have differing LSB values.

The operation of the individual valves in the valve banks 112 is controlled by control module 152 using input received from a user (not shown) via user interface 156. The control module 152 typically includes a microprocessor and memory, and the user interface 156 includes tactile, voice-activated, and/or graphical sets of inputs and outputs to receive user commands and provide appropriate feedback to the user.

The control module 152 can control the valve banks to alter any desired set of ventilation parameters selected by the user, such as the maximum pressure and/or volume of the gas 120 provided to the patient 136, the composition of the gas 120 (e.g., Fi02), and the shapes of trajectory waveforms. A trajectory waveform refers to the behavior of a selected ventilation parameter as a function of time (e.g., gas flow trajectory, Fi02 trajectory, and the like).

In one configuration, the control module 152 uses feedback from various sensors to control dynamically the ventilator system 100. The dashed lines show the feedback and control signal lines to and from the control module 152. Feedback signals are received from the flow meter 128 and pressure transducer 144. The pressure sensed by the pressure transducer is used to determine the pressure drop across the valve banks 112a-n. The pressure drop is used to control pressure regulator settings to provide a desired pressure in the mixing zone 116. Feedback signals from the selected gas component(s) sensor 124 may or may not be used to control operation of the valve banks 112. As will be appreciated, the sensor 124 will typically monitor the concentration of molecular oxygen in the gas 120, and the controller may use this signal for alarming. The control lines extend from the control module 152 to the first, second, . . . nth valve banks 112a-n and the first, second, . . . nth gas regulators 108a-b.

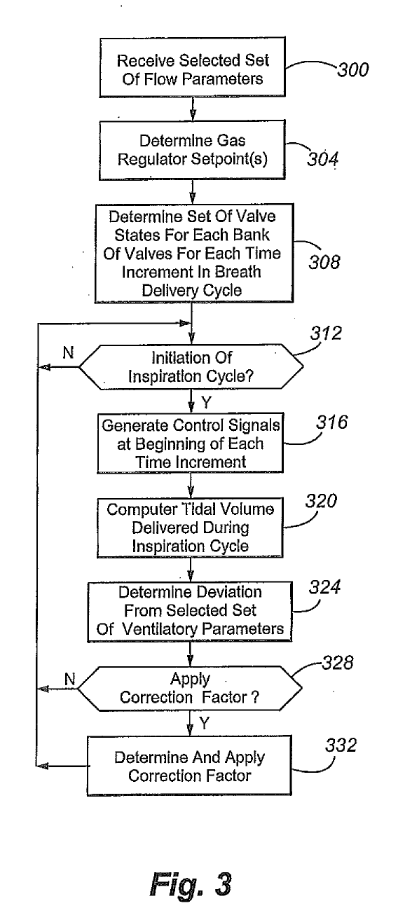

The operation of the control module 152 according to an embodiment of the present invention will now be discussed with reference to FIG. 3.

In step 300, the control module 152 receives, via the user interface 156, a selected set of flow parameters. Commonly, the flow parameters will vary depending on whether the breath is pressure or volume targeted. In a pressure targeted ventilator system, the control module 152 controls the gas flows through the orifices to realize a desired pressure versus time trajectory. In contrast in a volume targeted ventilator system, the module 152 controls the gas flows through the orifices to realize, for a selected inspiration cycle, a desired tidal volume of gas for delivery to the patient 136. For a pressure targeted breath, the user may set the target pressure for the gas 120, the inspiratory time (or the time interval over which the gas 120 is to be provided), and the rise time of the breath (which determines how quickly the ventilator system 100 arrives at the targeted pressure). For a volume targeted breath, the user commonly sets the tidal volume and a combination of inspiratory time, the inspiratory flow rate of the gas 120, the respiratory rate, and the ratio of inspiration to expiration time (I/E ratio), or the like. These parameters define the trajectory waveform to be employed.

In step 304, the control module 152 determines the gas regulator 108a-n setpoints. The setpoints are a function of the pressure of the gas 120 to be provided to the patient 136 and the pressure drop over the valve banks 112a-n.

In step 308, the control module 152 determines, for each time interval in the breath delivery cycle, a set of valve states for each valve bank. In an exemplary implementation in which the first gas source 104a is molecular oxygen and the second gas source 104b is air, the total flow trajectory (F_{TOTAL}) is split proportionately into air flow rate trajectory (F_{AIR}) and molecular oxygen flow rate trajectory (F_{OXYGEN}) based on the flow and Fi02 trajectories received from the user. For example assuming that the composition of the first gas source 104a is 78 mole % nitrogen, 21 mole % molecular oxygen, and 1 mole % argon, F_{TOTAL}is provided by the following equations:

F_{TOTAL}=F_{AIR}+F_{OXYGEN}

F_{AIR}=F_{TOTAL}×(1−Fi02)/0.79

F_{OXYGEN}=F_{TOTAL}×(Fi02−0.21)/0.79

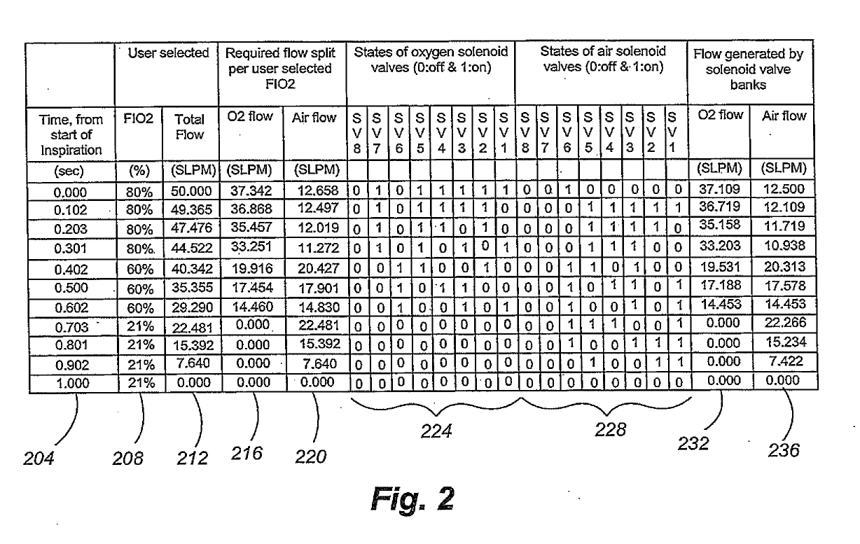

FIG. 2 is an example of a portion of a table 200 stored in the memory of the control module 152. It will be appreciated by those skilled in the art that the values in FIG. 2 are merely examples, and alternative values may be used in various embodiments of the present invention. The table can be configured as a look up table or determined dynamically. The table corresponds to a particular set of first and second regulator 108a,b set points and is used to select combinations of valves to be actuated during an inspiration cycle to generate the target trajectories of air and/or oxygen flow rates. Moving from left to right, the first column 204 is the time (seconds) from the start of the patient inspiration cycle, the second and third columns 208 and 212 are the user selected parameters Fi02 (percent) and total flow (SLPM), respectively, the fourth and fifth columns 216 and 220 are the required (ideal) flow split, based on the selected Fi02, for molecular oxygen and air flows (SLPM), respectively, the sixth and seventh columns 224 and 228 are the various binary valve states for the valves 148a-m in the first and second valve banks 112a-b, respectively, during selected time intervals of the cycle (with “0” being off (or closed) and “1” being on (or open) as shown or vice versa), and the eighth and ninth columns 232 and 236 are the particular (actual) flows (SLPM) generated by each valve bank 112a-b, with the eighth column 232 being the actual flow generated by the first gas source 104a and the ninth column 236 being the actual flow generated by the second gas source 104b.

In the example of FIG. 2, the user has selected (a) an Fi02 of 80% for the first 0.401 seconds of the inspiration cycle, 60% for the time period from 0.402 to 0.702 seconds, and 21% for the period from 0.703 seconds to 1.00 seconds and (b) a total flow of 50.000 SLPM for the first 0.101 seconds of the inspiration cycle, 49.365 SLPM for the time period from 0.102 to 0.202 seconds, 47.476 SLPM for the period from 0.203 to 0.300 seconds, 44.522 SLPM for the period from 0.301 to 0.401 seconds, 40.342 SLPM for the period from 0.402 to 0.499 seconds, 35.355 SLPM for the period from 0.500 to 0.601 seconds, 29.290 SLPM for the period from 0.602 to 0.702 seconds, 22.481 SLPM for the period from 0.703 to 0.800 seconds, 15.392 SLPM for the period from 0.801 to 0.901 seconds, and 7.640 SLPM for the period from 0.902 to 0.999 seconds. These variables can be selected manually by the user or generated using default trajectory profiles based on various user inputs, such as a user inputted Fi02, total flow, inspiratory time, and the like.

With reference to columns 204, 216, and 232, it can be seen that the first valve bank 112a provides decreasing levels of molecular oxygen flow until 0.702 seconds, after which point the molecular oxygen flow drops to zero. The decreasing flow is represented by differing sets of valves being opened in differing time intervals. For example, in the first time interval from 0.000 to 0.101 seconds, valves SV7 and SV5 to SV1 are opened in the oxygen valve bank, and the remaining oxygen valve bank valves are closed. In the second time interval from 0.102 to 0.203 seconds, valves SV7 and SV5 to SV2 are opened in the oxygen valve bank, with the remaining oxygen valve bank valves being closed.

With reference to columns 204 220 and 236, the air flow provided by the second valve bank 112b fluctuates over time. The highest air flow in the example shown is 22.266 SLPM at the time interval from 0.703 to 0.800 seconds. During this interval, valves SV6-SV4 and SV1 are opened in the air valve bank, and the remaining air valve bank valves are closed. The lowest air flow is 7.422 SLPM at the time interval from 0.902 to 0.999 seconds. During this interval, air valve bank valves SV5 and SV2-SV1 are opened, and the remaining air valve bank valves are closed.

In decision diamond 312, the control module 152 determines whether an inspiration cycle has been initiated. This can be done, for example, based on patient respiratory effort, timing signals generated as a result of a selected breathing frequency, or combinations thereof. Patient respiratory effort can be determined based on pressure and/or gas flow time dependent waveforms.

When an inspiration cycle is initiated, the control module 152, in step 316, generates and sends suitable sets of control signals at the beginning of each time interval in the inspiratory time period.

After an inspiration cycle is over, the control module 152, in step 320, computes the tidal volume delivered during the inspiration cycle (e.g., based on the total gas flow trajectory defined by the eighth and ninth columns 232 and 236) and, in step 324, determines the deviation, if any, from the selected set of ventilation parameters (e.g., the total gas flow defined by the total gas flow trajectory of the third column 212).

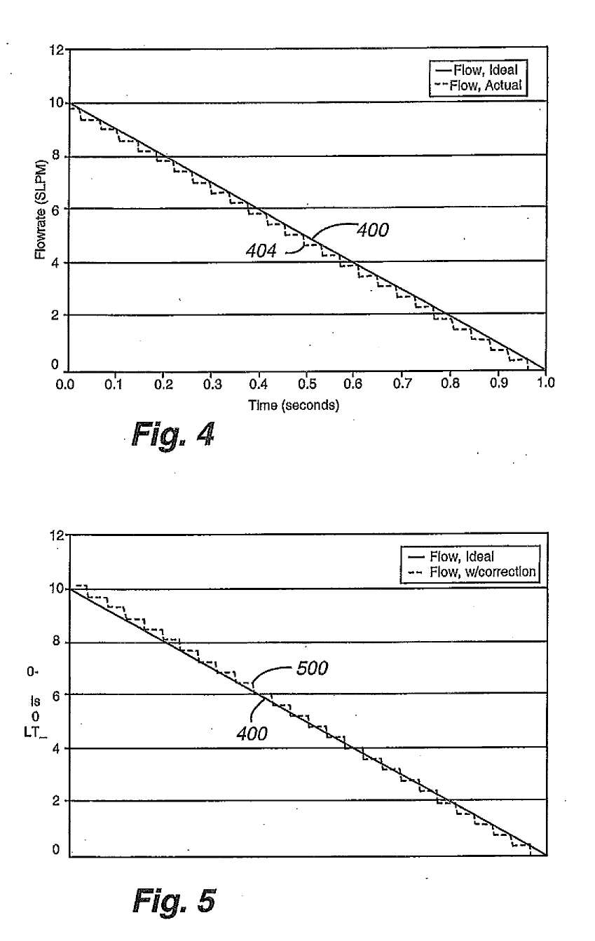

FIG. 4 is an example of a flow trajectory generated by the ventilation system 100 and shows the deviation determined in step 324. FIG. 4 shows target and delivered trajectories 400 and 404, respectively. The peak flow is 10 SLPM and the target trajectory 400 is a straight-line or linear profile. As will be appreciated, other trajectory profiles may be employed, such as curvilinear profiles. The delivered flow trajectory 404 has the appearance of a staircase profile. In some embodiments, the steps correspond to the time intervals in column 204 of FIG. 2. The area under a trajectory indicates the tidal volume delivered during inspiration. As can be seen from FIG. 4, the tidal volume delivered is lower than expected when compared to the target trajectory.

In decision diamond 328, the control module 152 determines whether a correction factor needs to be applied to the inspiratory time and/or one or more time interval(s) before the next inspiration cycle. This can be done, for example, by determining the level of significance of the deviation, with only significant deviations warranting application of a correction factor. In one configuration, whether a deviation is significant is based on a comparison of the deviation against a selected threshold value. If the deviation exceeds the threshold value, it is considered to be significant; if not, it is not considered to be significant. As will appreciated, significance can be defined by other suitable mathematical techniques, depending on the application.

When a correction factor is to be applied, the control module 152, in step 332, determines and applies a suitable correction factor. In one configuration, the correction factor is defined as the target tidal volume divided by actual tidal volume. FIG. 5 shows the delivered flow trajectory 500 for a subsequent (next) inspiration cycle after application of the correction factor. Comparing FIG. 5 with FIG. 4, it can be seen that the deviation between targeted and delivered trajectories is much smaller. Specifically, for the depicted example, the deviation in tidal volume before correction is −3.88% and after correction is 0.159%.

When no correction is to be applied or after step 332, the control module 152 returns to decision diamond 312.

Returning to FIG. 1, another embodiment will now be discussed. In this embodiment, the valves 148 in the first, second, . . . nth valve banks 112a-n are operated under a choked flow condition to generate the desired flow trajectory. Choked flow occurs when the velocity of gas through an orifice is at least a sonic velocity. Subsonic gas velocities through an orifice do not produce choked flow conditions.

Under choked flow conditions, the mass flow rate through the valve orifices depends on upstream pressure as shown by the following equation:

$$Q = CAP \sqrt{( \frac{kM}{ZRT} ) {( \frac{2}{k + 1} )}^{( k + 1 ) / ( k - 1 )}}$$

where: Q=mass flow rate; C=discharge coefficient; A=orifice cross-sectional area; P=upstream pressure; k=cₚ/c_{V}of the gas; M=gas molecular mass; Z=gas compressibility factor at P and T; R=Universal gas law constant; T=absolute gas temperature; cₚ=specific heat of the gas at constant pressure; and c_{V}=specific heat of the gas at constant volume. As can be seen from this equation, under choked flow conditions the mass flow rate is independent of the pressure downstream of the orifice.

The upstream pressure is controlled to maintain choked flow conditions by controlling the pressure set points on the regulators 108a-n. Choked flow typically occurs when the ratio of absolute pressure downstream of an orifice relative to the absolute pressure upstream of the orifice is 0.528 or less. Variations in pressure downstream of the orifice which do not cause this ratio to be exceeded will generally not change the rate of flow through the orifice.

By maintaining the downstream and upstream pressures at the 0.528 ratio or below, changes not only in the effective (open) orifice area in a valve bank 112 but also in the pressure set points can be correlated precisely to a resulting change in flow rate of the gas 120, regardless of the downstream flow conditions. These properties can enable a common ventilator system 100 to serve both adult patients and infant patients. For example, an adult ventilator capable of delivering peak flow of about 100 SLPM or more can be made into an infant ventilator capable of delivering a peak flow of about 40 SLPM or less while increasing the accuracy of the flow/tidal volume delivered simply by setting the upstream pressure of each gas to a different level (e.g., which, for an original peak flow of 100 SLPM, is 40% of the original setting to produce a peak flow of 40 SLPM).

The upstream pressure, or pressure set points, in each of the first, second, . . . nth gas regulators 108a-n can be the same or different, depending on the application. In either case, a combination of upstream pressures, or pressure set points, correspond to a specific set of flow and valve state relationships as shown in FIG. 2. That is, for a given set of user selected parameters multiple tables will exist, with each table corresponding to specific combinations of pressure set points.

The appropriate mass flow rate Q and pressure set points to be employed depend on the lung capacity of the patient 136. To determine which pressure set points to use, the control module 152 uses patient lung capacity measures input by the user. Examples of such measures include total lung capacity, vital capacity, and tidal volume. These measures can be estimated based on the gender and height and/or the ideal body weight of the patient.

A number of variations and modifications of the invention can be used. It would be possible to provide for some features of the invention without providing others.

For example in one alternative embodiment, all or part of the valve banks in the system 100 are not operated under choked flow conditions.

In another alternative embodiment, the valve banks are replaced by single choked flow orifices. Flow rate is changed by changing the upstream pressure.

In yet another alternative embodiment, the control module 152 is in the form of a number of distributed or satellite controllers to perform specific or limited functions.

The foregoing discussion of the invention has been presented for purposes of illustration and description. Further, the description is not intended to limit the invention to the form disclosed herein. Consequently, variations and modifications commensurate with the above teachings, within the skill or knowledge of the relevant art, are within the scope of the present invention. The embodiments described hereinabove are further intended to explain the best mode presently known of practicing the invention and to enable others skilled in the art to utilize the invention in such or in other embodiments and with the various modifications required by their particular application or use of the invention. It is intended that the appended claims be construed to include alternative embodiments to the extent permitted by the prior art.

What is claimed is:

1. A method, comprising:
providing a ventilation system for receiving at least one input gas, the ventilation system comprising at least one valve bank to meter a flow of the at least one input gas and deliver an output gas to a patient, the at least one valve bank comprising a plurality of valves with each valve comprising a respective orifice;
receiving a set of ventilation parameters;
based on the set of ventilation parameters, determining, for each of a plurality of successive time intervals in an inspiration cycle, a respective plurality of operating states for selected valves in the at least one valve bank to provide the output gas, the output gas having at least one of a selected gas composition and flow trajectory;
when an inspiration cycle is initiated, implementing, for each successive time interval, the determined operating states for the selected valves in the at least one valve bank.

2. The method of claim 1, wherein the at least one input gas is received from at least one gas source and the at least one gas source comprises at least first and second gas sources, wherein the at least one valve bank comprises a first valve bank corresponding to the first gas source and a second valve bank corresponding to the second gas source, and wherein the first and second valve banks are positioned upstream of a mixing zone for the first and second gases.

3. The method of claim 1, wherein the at least one of a gas composition and flow trajectory is gas composition trajectory, wherein each of the valves in the at least one valve bank has binary operating states, and wherein, for a selected maximum flow rate Y, a smallest flow rate X for any valve in the at least one valve bank is provided by the equation:
X=Y/2^{m},
where m is the number of valves in the at least one valve bank.

4. The method of claim 1, wherein the at least one input gas is received from at least one gas source and the at least one gas source comprises first and second gas sources, the first gas source comprising predominantly molecular oxygen and the second gas source comprising predominantly air, wherein the at least one valve bank comprises a first valve bank corresponding to the first gas source and a second valve bank corresponding to the second gas source, wherein, for a selected input gas pressure, a first valve in the first valve bank has a first flow rate, the first flow rate being lower than flow rates of other valves in the first valve bank, wherein, for the selected input gas pressure, a second valve in the second valve bank has a second flow rate, the second flow rate being lower than flow rates of other valves in the second valve bank, wherein, for the selected input gas pressure, the first and second flow rates are different, and wherein, for the selected input gas pressure, at least two valves in the first valve bank have differing flow rates and at least two valves in the second valve bank have differing flow rates.

5. The method of claim 3, wherein, in the at least one valve bank, at least one valve is open during a first time interval and closed during a second time interval, wherein, for a selected input gas pressure, a plurality of valves in the at least one valve bank have different flow rates, and wherein the different flow rates are multiples of X.

6. The method of claim 2, wherein the set of ventilation parameters comprise a plurality of target pressure for the output gas provided to the patient, an inspiratory time, a rise time, tidal volume, inspiratory flow rate, respiratory rate, ratio of inspiration to expiration time, and FiO2 and wherein a number of valves in the first valve bank is different from a number of valves in the second valve bank.

7. The method of claim 1, further comprising:
after the inspiration cycle is completed, comparing at least one of a target tidal volume and a target trajectory with at least one of an actual tidal volume and an actual trajectory provided to the patient in the implementing step to determine a deviation;
determining whether the deviation is significant; and
when the deviation is significant, applying a correction factor to at least one of the time intervals, wherein the correction factor is the target tidal volume divided by the actual tidal volume.

8. The method of claim 1, wherein, in the implementing step, the valves in the at least one valve bank are operated in a choked flow condition and wherein the ventilation system comprises at least one gas regulator to regulate an input gas pressure upstream of the at least one valve bank.

9. A ventilator, comprising:
at least one valve bank to meter a flow of at least one input gas and deliver an output gas for patient inhalation, the at least one valve bank comprising a plurality of valves with each valve comprising a respective orifice; and
a control module operable to determine, for each of a plurality of successive time intervals, a respective plurality of differing operating states for at least one of the valves in the at least one valve bank and, during an inspiration cycle, provide control signals to implement, for each successive time interval, the determined operating states for the at least one valve to provide the output gas.

10. The ventilator of claim 9, wherein the at least one input gas is received from at least one gas source and the at least one gas source comprises at least first and second gas sources, wherein the at least one valve bank comprises a first valve bank corresponding to the first gas source and a second valve bank corresponding to the second gas source, and wherein the first and second valve banks are positioned upstream of a mixing zone for the first and second gases.

11. The ventilator of claim 9, wherein the at least one valve bank provides a gas composition trajectory, wherein each of the valves in the at least one valve bank is a two-way solenoid valve, and wherein, for a selected maximum flow rate Y, a smallest flow rate X for any valve in the at least one valve bank is provided by the equation:
X=Y/2^{m},
where m is the number of valves in the at least one valve bank, wherein, for a selected input gas pressure, a plurality of valves in the at least one valve bank have different flow rates, and wherein the different flow rates are multiples of X.

12. The ventilator of claim 10, wherein, for a selected input gas pressure, at least two valves in the first valve bank have differing flow rates and at least two valves in the second valve bank have differing flow rates, wherein a first valve in the first valve bank has a first flow rate, the first flow rate being lower than flow rates of other valves in the first valve bank, wherein a second valve in the second valve bank has a second flow rate, the second flow rate being lower than flow rates of other valves in the second valve bank, and wherein, for the selected input gas pressure, the first and second flow rates are different.

13. The ventilator of claim 9, wherein the valves in the at least one valve bank are operated in a choked flow condition and further comprising at least one gas regulator to regulate a gas pressure upstream of the at least one valve bank.

14. A method, comprising:
providing a ventilator to receive at least one input gas from at least one gas source and deliver an output gas for patient inhalation, the ventilator comprising at least one gas regulator to control a pressure of the at least one input gas and at least one valve positioned downstream of the gas regulator, wherein the at least one valve comprises an orifice and the output gas is derived from the at least one gas source; and
while maintaining the at least one valve at choked flow, varying the input gas pressure to provide differing output gas flow rates

15. The method of claim 14 wherein the different output gas flow rates are adapted for use with patients having differing lung conditions.

16. The method of claim 14, wherein the varying step comprises:
selecting a first flow rate of the output gas during an inspiratory cycle by a first patient;
during the inspiratory cycle by the first patient, maintaining, by the at least one gas regulator, a first input gas pressure, wherein, at the first input gas pressure, the orifice of the at least one valve operates at choked flow;
selecting a second flow rate of the output gas during an inspiratory cycle by a second patient, the first and second patients having differing lung capacities and the first and second flow rates being different; and
during the inspiratory cycle by the second patient, maintaining, by the at least one gas regulator, a second input gas pressure, wherein, at the second input gas pressure, the orifice of the at least one valve operates at choked flow.

17. The method of claim 16, wherein a ratio of the output gas pressure to the input gas pressure is 0.528 or less and wherein the first patient is an adult and the second patient is an infant.

18. The method of claim 16, wherein a peak flow for the first patient is at least about 75 SLPM and a peak flow for the second patient is no more than about 40 SLPM.

19. The method of claim 16, wherein the at least one gas source comprises at least first and second gas sources, wherein the at least one valve comprises a first valve bank corresponding to the first gas source and a second valve bank corresponding to the second gas source, wherein the first and second valve banks are positioned upstream of a mixing zone for the first and second gases, and further comprising:
receiving a set of ventilation parameters;
based on the set of ventilation parameters, determining, for each of a plurality of successive time intervals in an inspiration cycle, a respective plurality of operating states for each valve in each of the first and second valve banks to provide at least one of a selected gas composition and flow trajectory;
when an inspiration cycle is initiated, implementing, for each successive time interval, the determined operating states for each valve in each of the first and second valve banks.

20. A ventilator to provide an output gas for patient inhalation, the ventilator comprising:
at least one gas regulator to control a pressure of at least one input gas;
at least one valve positioned downstream of the gas regulator, wherein the at least one valve comprises an orifice and the output gas is derived from the at least one input gas; and
a control module operable to vary the input gas pressure to provide differing output gas flow rates for differing patients while maintaining the at least one valve at choked flow.

21. The ventilator of claim 20, wherein the differing patients have differing lung capacities and wherein the control module is adapted to perform the following operations:
select a first flow rate of the output gas during an inspiratory cycle by a first patient;
during the inspiratory cycle by the first patient, maintain, by the at least one gas regulator, a first input gas pressure, wherein, at the first input gas pressure, the orifice of the at least one valve operates at choked flow;
select a second flow rate of the output gas during an inspiratory cycle by a second patient, the first and second patients having differing lung capacities and the first and second flow rates being different; and
during the inspiratory cycle by the second patient, maintain, by the at least one gas regulator, a second input gas pressure, wherein, at the second input gas pressure, the orifice of the at least one valve operates at choked flow.

22. The ventilator of claim 21, wherein a ratio of the output gas pressure to the input gas pressure is 0.528 or less and wherein the first patient is an adult and the second patient is an infant.

23. The ventilator of claim 21, wherein a peak flow for the first patient is at least about 75 SLPM and a peak flow for the second patient is no more than about 40 SLPM.

24. The ventilator of claim 20, wherein the at least one valve is a plurality of valves, wherein the control module is further operable to determine, for each of a plurality of successive time intervals, a respective plurality of differing operating states for at least one of the valves and, during an inspiration cycle, provide control signals to implement, for each successive time interval, the determined operating states for the at least one valve.
